(19) United States
(12) Patent Application Publication
(10) Pub. No.: US 2016/0375179 A1
(43) Pub. Date: Dec. 29, 2016

(54) PROCESS OF MAKING SCAFFOLD WITH INTERFACE TO PROMOTE COATING ADHESION

(71) Applicant: Abbott Cardiovascular Systems Inc., Santa Clara, CA (US)

(72) Inventors: Jose R. Gamez, Menlo Park, CA (US); Karen Wang, Cupertino, CA (US); Mary Beth Kossuth, San Jose, CA (US); Joel Harrington, Redwood City, CA (US); James P. Oberhauser, Saratoga, CA (US)

(21) Appl. No.: 14/750,913

(22) Filed: Jun. 25, 2015

(51) Int. Cl. A61L 31/06; A61F 2/915; A61L 31/16; A61F 2/844

(52) U.S. Cl. CPC A61L 31/06; A61F 2/844; A61F 2/915; A61L 31/16; A61F 2210/0004; A61F 2210/0076; A61F 2230/0069; A61F 2250/0067; A61F 2002/91558; A61L 2300/416; A61L 2420/02; A61L 2420/08

(57) ABSTRACT

Methods of fabricating a stent are disclosed including forming a primer layer on a surface of the scaffold including a first polylactide polymer. The primer layer includes a second polylactide polymer and is free of a therapeutic agent. The scaffold with the primer layer is thermally treated to condition the scaffold. A therapeutic layer is formed over the primer layer and the therapeutic layer includes the second polylactide polymer and a drug. The scaffold is crimped and the primer layer improves adhesion of the therapeutic layer to the scaffold and reduces or prevents damage to the therapeutic layer during crimping.

### BACKGROUND OF THE INVENTION

Field of the Invention

This invention relates polymeric medical devices, in particular, bioresorbable stents or scaffolds including polymer and drug coatings.

Description of the State of the Art

This invention relates to radially expandable endoprostheses that are adapted to be implanted in a bodily lumen. An “endoprosthesis” corresponds to an artificial device that is placed inside the body. A “lumen” refers to a cavity of a tubular organ such as a blood vessel. A stent is an example of such an endoprosthesis. Stents are generally cylindrically shaped devices that function to hold open and sometimes expand a segment of a blood vessel or other anatomical lumen such as urinary tracts and bile ducts. Stents are often used in the treatment of atherosclerotic stenosis in blood vessels. “Stenosis” refers to a narrowing or constriction of a bodily passage or orifice. In such treatments, stents reinforce body vessels and prevent restenosis following angioplasty in the vascular system. “Restenosis” refers to the reoccurrence of stenosis in a blood vessel or heart valve after it has been treated (as by balloon angioplasty, stenting, or valvuloplasty) with apparent success.

Stents are typically composed of a scaffold or scaffolding that includes a pattern or network of interconnecting structural elements or struts, formed from wires, tubes, or sheets of material rolled into a cylindrical shape. This scaffolding gets its name because it possibly physically holds open and, if desired, expands the wall of the passageway. Typically, stents are capable of being compressed or crimped onto a catheter so that they can be delivered to and deployed at a treatment site.

Delivery includes inserting the stent through small lumens using a catheter and transporting it to the treatment site. Deployment includes expanding the stent to a larger diameter once it is at the desired location. Mechanical intervention with stents has reduced the rate of restenosis as compared to balloon angioplasty. Yet, restenosis remains a significant problem. When restenosis does occur in the stented segment, its treatment can be challenging, as clinical options are more limited than for those lesions that were treated solely with a balloon.

Stents are generally made to withstand the structural loads, namely radial compressive forces, imposed on the scaffold as it supports the walls of a vessel. Therefore, a stent must possess adequate radial strength if its function is to support a vessel at an increased diameter. Radial strength, which is the ability of a stent to resist radial compressive forces, relates to a stent's radial yield strength and radial stiffness around a circumferential direction of the stent. A stent's “radial yield strength” or “radial strength” (for purposes of this application) may be understood as the compressive loading or pressure, which if exceeded, creates a yield stress condition resulting in the stent diameter not returning to its unloaded diameter, i.e., there is irrecoverable deformation of the stent.

Some treatments with stents require its presence for only a limited period of time. Once treatment is complete, which may include structural tissue support and/or drug delivery, it may be desirable for the stent to be removed or disappear from the treatment location. One way of having a stent disappear may be by fabricating a stent in whole or in part from materials that erode or disintegrate through exposure to conditions within the body. Stents fabricated from biodegradable, bioabsorbable, bioresorbable, and/or bioerodable materials such as bioabsorbable polymers can be designed to completely erode only after the clinical need for them has ended.

Stents are used not only for mechanical intervention but also as vehicles for providing biological therapy. Biological therapy uses medicated stents to locally administer a therapeutic substance. A medicated stent may be fabricated by coating the surface of either a metallic or polymeric scaffold with a polymeric carrier that includes an active or bioactive agent or drug. Polymeric scaffolds may also serve as a carrier of an active agent or drug. An active agent or drug may also be included on a scaffold without being incorporated into a polymeric carrier.

One challenge with a therapeutic coating on a stent is reducing or preventing damage or defects to the coating during manufacturing and use of the stent. Once formed, on a scaffold or a stent body, a coating is subjected to external forces as well as forces arising from deformation of the scaffold itself. A coating is susceptible to damage or loss of adhesion arising from such forces. The susceptibility of a coating to damage or defects is higher when there is poor adhesion between the coating and the scaffold. Poor adhesion generally is highest between coating materials and scaffold materials that are incompatible, such as between metals and polymers.

### INCORPORATION BY REFERENCE

All publications, patents, and patent applications mentioned in this specification are herein incorporated by reference to the same extent as if each individual publication, patent, or patent application was specifically and individually indicated to be incorporated by reference, and as if each said individual publication, patent, or patent application was fully set forth, including any figures, herein.

### SUMMARY OF THE INVENTION

An aspect of the present invention includes a method of fabricating a stent comprising: providing a scaffold comprising a polymer formulation including a first polylactide polymer; forming a primer layer on a surface of the scaffold, wherein the primer layer comprises a second polylactide polymer and the primer layer is free of a therapeutic agent and; thermally treating the scaffold with the primer layer to condition the scaffold; forming a therapeutic layer over the primer layer, wherein the therapeutic layer comprises the second polylactide polymer and a drug; and crimping the scaffold having the therapeutic and primer layers from a fabricated diameter to a reduced diameter for delivery into a vascular lumen, wherein the primer layer improves adhesion of the therapeutic layer to the scaffold and reduces or prevents damage to the therapeutic layer. The above aspect include one or more of the following aspects in any combination: wherein the therapeutic layer comprises damage over less than 2% of a surface area of an outer surface of the crimped stent and wherein the damage comprises flaps, tears, bare spots, and/or peeling; wherein the crimping comprises applying a radial inward force on an outer surface of the stent with metallic surfaces to reduce a diameter of the stent to the reduced diameter; wherein to condition the scaffold comprises modification of scaffold properties that include a decrease in density of the scaffold polymer, an increase in elongation at break of the scaffold polymer, a decrease in modulus of the scaffold polymer, an increase in radial strength of the scaffold, an increase in expansion capability of the scaffold, a reduction in damage to the scaffold at crimping, or any combination thereof; wherein the polymer formulation is selected from the group consisting of poly(L-lactide) (PLLA), a copolymer of PLLA and polycaprolactone, a blend of PLLA and a copolymer of PLLA and PCL; wherein the polymer formulation comprises a total CL content of 2 to 8 wt %; wherein the thermal treatment comprises increasing a temperature of the stent above a glass transition temperature (Tg) and below a melting temperature (Tm) of the polymer formulation followed by reducing the temperature below the Tg; wherein the thermal treatment comprises increasing the temperature of the scaffold to 10 to 20 deg C. above the Tg of the polymer formulation and maintaining the increased temperature for 10 to 20 minutes or 5 to 10 minutes followed by reducing the temperature to below the Tg; wherein the second polylactide polymer is 50/50 poly(DL-lactide); wherein the drug is selected from the group consisting of everolimus, rapamycin, novolimus, zotarolimus, and biolimus; and wherein a thickness of the primer layer is 0.2 to 2 microns and a thickness of the therapeutic layer is 1 to 3 microns.

An aspect of the invention includes a method of fabricating a stent comprising: providing a scaffold comprising a polymer formulation including a first polylactide polymer; forming a primer layer on a surface of the scaffold, wherein the primer layer comprises a second polylactide polymer and a drug and the drug is 0.1 to 10 wt % of the primer layer; thermally treating the scaffold with the primer layer to condition the scaffold; forming a therapeutic layer over the primer layer, wherein the therapeutic layer comprises the second polylactide polymer and a drug and the drug is greater than 20 wt % of the therapeutic layer; and crimping the scaffold having the primer and therapeutic layers from a fabricated diameter to a reduced diameter for delivery into a vascular lumen, wherein the primer layer improves adhesion of the therapeutic layer to the scaffold and reduces or prevents damage to the therapeutic layer. The above aspect include one or more of the following aspects in any combination: wherein the drug is 40 to 60 wt % of the therapeutic layer; wherein the therapeutic layer comprises damage over less than 2% of a surface area of an outer surface of the crimped stent and wherein the damage comprises flaps, tears, bare spots, and/or peeling; wherein the crimping comprises applying a radial inward force on an outer surface of the stent with metallic surfaces to reduce a diameter of the stent to the reduced diameter; wherein to condition the scaffold comprises modification of scaffold properties that include a decrease in density of the scaffold polymer, an increase in elongation at break of the scaffold polymer, a decrease in modulus of the scaffold polymer, an increase in radial strength of the scaffold, an increase in expansion capability of the scaffold, a reduction in damage to the scaffold at crimping, or any combination thereof; wherein the polymer formulation is selected from the group consisting of poly(L-lactide) (PLLA), a copolymer of PLLA and polycaprolactone, a blend of PLLA and a copolymer of PLLA and PCL; wherein the polymer formulation comprises a total CL content of 2 to 8 wt %; wherein the thermal treatment comprises increasing a temperature of the stent above a glass transition temperature (Tg) and below a melting temperature (Tm) of the polymer formulation followed by reducing the temperature below the Tg; wherein the thermal treatment comprises increasing the temperature of the scaffold to 10 to 20 deg C. above the Tg of the polymer formulation and maintaining the increased temperature for 10 to 20 minutes or 5 to 10 minutes followed by reducing the temperature to below the Tg; wherein the second polylactide polymer is 50/50 poly(DL-lactide); wherein the drug is selected from the group consisting of everolimus, rapamycin, novolimus, zotarolimus, and biolimus; and wherein a thickness of the primer layer is 0.2 to 2 microns and a thickness of the therapeutic layer is 1 to 3 microns.

An aspect of the invention includes a method of fabricating a stent comprising: providing a scaffold comprising a polymer formulation including a scaffold polylactide polymer; forming a primer layer on a surface of the scaffold, wherein the primer layer comprises a primer polylactide polymer and a drug and the drug is less than 10 wt % of the primer layer; thermally treating the scaffold with the primer layer to condition the scaffold; forming a therapeutic layer over the primer layer, wherein the therapeutic layer comprises a therapeutic polylactide polymer and a drug and the drug is greater than 20 wt % of the therapeutic layer; and crimping the scaffold having the primer and therapeutic layers from a fabricated diameter to a reduced diameter for delivery into a vascular lumen, wherein the first layer improves adhesion of the therapeutic layer to the scaffold and reduces or prevents crimping-induced damage to a surface of the therapeutic layer.

An aspect of the invention includes a method of fabricating a stent comprising: providing a stent body; forming a primer layer on a surface of the stent body, wherein the primer layer comprises a polylactide polymer and is free of a therapeutic agent; thermally treating the scaffold with the primer layer to condition the scaffold; forming a therapeutic layer over the primer layer, wherein the therapeutic layer comprises the polylactide polymer and a drug; and crimping the scaffold having the therapeutic and primer layers from a fabricated diameter to a reduced diameter for delivery into a vascular lumen, wherein the primer layer improves adhesion of the therapeutic layer to the scaffold and reduces or prevents crimping-induced damage to a surface of the therapeutic layer. The aspect may include any one or any combination of the following aspects. The stent body is metallic. The stent body is a bioabsorbable polymer formulation.

An aspect of the invention includes a stent comprising: a scaffold comprising a polymer formulation including a first polylactide polymer; a primer layer on a surface of the scaffold, wherein the primer layer comprises a second polylactide polymer and the primer layer is free of a therapeutic agent and; a therapeutic layer over the primer layer, wherein the therapeutic layer comprises the second polylactide polymer and a drug, wherein the scaffold is in a crimped configuration over a catheter at a reduced diameter for delivery into a vascular lumen, wherein the primer layer improves adhesion of the therapeutic layer to the scaffold and reduces or prevents damage to the therapeutic layer. The aspect may include one or more of the following aspects in any combination: wherein the therapeutic layer comprises damage over less than 2% of a surface area of an outer surface of the crimped stent, wherein the damage comprises flaps, tears, bare spots, and/or peeling; wherein the polymer formulation is selected from the group consisting of poly(L-lactide) (PLLA), a copolymer of PLLA and polycaprolactone, and a blend of PLLA and a copolymer of PLLA and PCL; wherein the polymer formulation comprises a total CL content of 2 to 8 wt %; wherein the second polylactide polymer is 50/50 poly(DL-lactide); wherein the drug is selected from the group consisting of everolimus, rapamycin, novolimus, zotarolimus, and biolimus, wherein a thickness of the primer layer is 0.2 to 2 microns; and wherein a thickness of the therapeutic layer is 1 to 3 microns.

### BRIEF DESCRIPTION OF THE DRAWINGS

FIG. 1 depicts an exemplary scaffold.

FIG. 2 depicts a cross-section of a stent surface with a polymer and drug layer.

FIG. 3 shows a cross-sectional view of blades of an iris-type crimper taken along the crimper axis when reducing a polymer scaffold diameter from a first, large diameter to a second, smaller diameter.

FIG. 4 depicts a cross-section of a strut of a scaffold with a primer layer over the strut and a therapeutic layer over the primer layer.

FIG. 5 depicts a deployed untreated stent at time=0, i.e., right after deployment.

FIG. 6 depicts a deployed treated stent.

FIG. 7 depicts a distal end of a crimped untreated stent having multiple areas where coating was compromised upon crimping.

FIG. 8 depicts a distal end of a crimped treated stent showing less coating damage than the untreated stent.

FIG. 9 depicts a middle section of an untreated stent with multiple areas where the coating was compromised upon crimping.

FIG. 10 depicts a middle section of a crimped treated stent showing less coating damage than the untreated stent.

FIG. 11 depicts an untreated stent showing a proximal rings section as crimped before final sheath is placed over the stent having multiple areas where coating has been compromised.

FIG. 12 depicts a proximal end of a crimped treated stent showing less coating damage than the untreated stent.

### DETAILED DESCRIPTION OF THE INVENTION

The present invention relates to processes for improving adhesion between a stent body and therapeutic coatings. In particular, the stent body is a scaffold composed of a bioabsorbable polymer and the therapeutic coating includes another bioabsorbable polymer carrier and a drug. The scaffold polymer and the coating polymer may be chemically similar and thus have high compatibility; however, the coating is susceptible to damage during manufacturing and use of the stent. The processes include forming an interface or layer between the scaffold and coating which reduces or prevents damage or defects due to loss of adhesion between scaffold and the coating.

In general, a radially expandable stent can have virtually any structural pattern that is compatible with a bodily lumen in which it is implanted. In certain aspects, a stent is composed of a pattern or network of circumferential rings and longitudinally extending interconnecting structural elements of struts or bar arms. In general, the struts are arranged in patterns, which are designed to contact the lumen walls of a vessel and to maintain vascular patency.

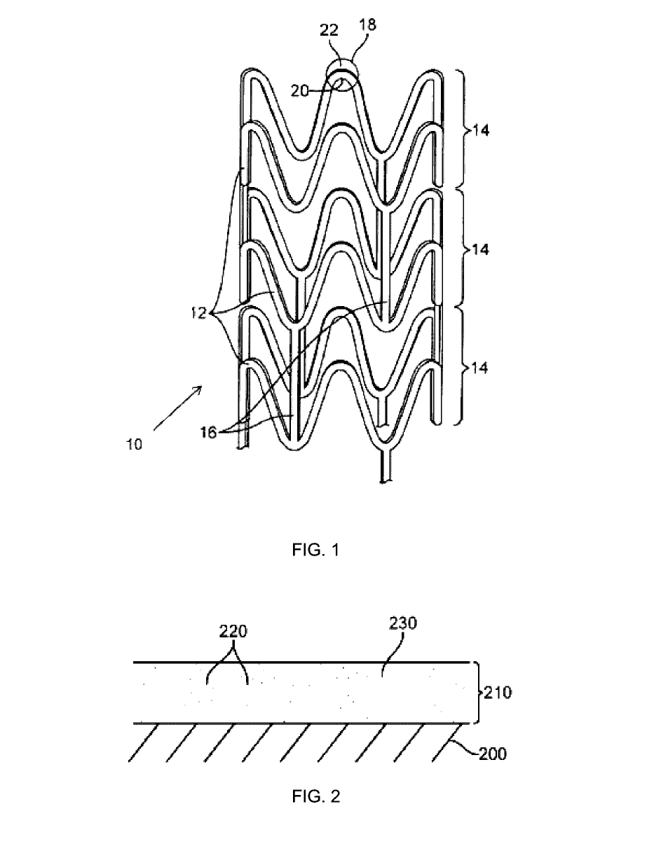

An exemplary structure of a stent body or scaffold is shown in FIG. 1. FIG. 1 depicts a stent 10 which is made up of struts 12. Stent 10 has interconnected cylindrical rings 14 composed of undulating struts. Cylindrical rings 14 are connected by linking struts or links 16. The embodiments disclosed herein are not limited to fabricating stents or to the stent pattern illustrated in FIG. 1. The embodiments are easily applicable to other stent patterns and other devices. The variations in the structure of patterns are virtually unlimited. The outer diameter of a fabricated stent (prior to crimping and deployment) may be between 0.2-5.0 mm. For coronary applications, a fabricated stent diameter is 2.5-5 mm. The length of the stents may be between about 6-30 mm or more depending on the application.

A stent body or scaffold can be made of a polymer or a metal. Metallic materials include stainless steel, cobalt chromium alloys, and nickel titanium alloys. Polymers can be biostable, bioabsorbable, biodegradable, bioresorbable, or bioerodable. Biostable refers to polymers that are not biodegradable. The terms biodegradable, bioabsorbable, bioresorbable, and bioerodable, as well as degraded, eroded, resorbed, and absorbed, are used interchangeably and refer to polymers that are capable of being completely eroded or absorbed when exposed to bodily fluids such as blood and can be gradually resorbed, absorbed, and/or eliminated by the body.

A polymer coating on the surface of a stent body or scaffold may also include a biodegradable polymer. The biodegradable polymer may be a carrier for an active agent or drug.

A radial thickness or thickness of the stent body or scaffold may be 80 to 100 microns, 90 to 110 microns, 100 to 120 microns, 120 to 140 microns, 140 to 160 microns, or greater than 160 microns.

The coating is typically much thinner than the struts of the scaffolding, for example, the coating can be 1 to 10 microns, 1 to 3 microns, 3 to 5 microns, or 5 to 10 microns. In general, it is desirable for the radial thickness to be as low as possible.

FIG. 2 depicts a cross-section of a stent surface with a polymer and drug coating layer 210 over a substrate 200. Coating layer 210 includes a drug 220 dispersed in a coating polymer 230. A substrate or scaffold can be metallic, polymeric, ceramic, or other suitable material.

A biodegradable stent may be fabricated from a tube with a thin wall initially having no holes or voids. The pattern of structural elements may be formed by laser machining. Material is removed from selected regions of tube which results in the pattern of structural elements.

The manufacturing process for a bioabsorbable stent may include several steps. A polymeric tube may be formed using melt processing such as extrusion or injection molding. Prior to laser machining, the tube may be processed to modify its mechanical properties that also improve stent properties such as radial strength and resistance to fracture. Such processes may include radially deforming the tube. The scaffold pattern may then be formed by laser machining. A therapeutic coating may be formed over the scaffold.

A polymer coating over a scaffold may be formed using various solution techniques which involve application of a coating composition including a polymer, drug, and solvent to the scaffold surface, followed by removing the solvent. The coating composition can be applied to a scaffold substrate by various methods, such as, dip coating, brushing, or spraying. The aspects of the present invention are not limited to any particular application or deposition technique. In particular, spray coating a stent typically involves mounting or disposing a stent on a support, followed by spraying a coating composition from a nozzle onto the mounted stent. Solvent is removed from the deposited coating composition to form the coating. There typically is some residual solvent remaining in the coating after the solvent removal or solvent removal steps. As discussed in more detail below, solvent removal can be performed through evaporation at room or ambient temperature or by heating or exposing a coated stent to a temperature above room temperature. Room or ambient temperature may be between 20 and 30 deg C. and any temperature or range in between.

If a coating layer of a target thickness (or mass) is formed with a single application step and then followed by solvent removal, the coating layer that results can be nonuniform, include coating defects, or both. Stents, particularly those for coronary use, comprise an intricate stent pattern with small dimensions. Too much coating applied all at once to load the desired amount of drug, the applied solution could form webs, pools, or strands in the stent pattern. Instead of the desired conformal coating, a highly non-uniform coating results. Therefore, a coating of a target thickness (or mass) is preferably formed with two or more cycles or passes of a coating composition application, such as spraying. After each cycle or pass, a solvent removal or drying step is performed. The solvent removal step after each pass is referred to as interpass drying. A cycle or pass refers to the application of a coating composition without an intervening solvent removal step, such as blowing warm air on the stent. In spraying, a cycle or pass can include directing the spray plume over the length of a stent one or more times. After each coating composition application pass, the application of coating composition on the substrate is stopped, which is followed by interpass solvent removal. An exemplary coating process is described in US 2010/0323093.

The above processes are typically performed with the scaffold at a diameter larger than that required for delivery into vessel. After coating, the coated scaffold may be reduced in diameter or crimped to a diameter suitable for delivery over a support such a delivery balloon. The crimped scaffold may then be subjected to a sterilization process such as e-beam radiation. The stent is implanted in a patient by positioning the crimped scaffold at a site of stenosis in a blood vessel and expanding the stent with the delivery balloon.

A radially expandable scaffold or stent body should have the ability to hold open narrowed portions of blood vessels. Therefore, the scaffold should possess a radial strength in an expanded state that is sufficiently high and sustainable to maintain the expanded vessel size for a period of weeks or months. A polymer or polymer formulation for a scaffold should be stiff and strong after processing into a scaffold under physiological conditions within a human body. Polymer or polymer formulations that have a glass transition temperature (Tg) in a dry state sufficiently above human body temperature (approximately 37 deg C.), particularly those that include semicrystalline polymers, meet the above criterial. Polylactide and polylactide based polymers such as poly(L-lactide) (PLLA) are examples of such polymers.

The polymer or polymer formulation of a scaffold of the present invention may include polylactide-based polymers such as, but are not limited to poly(L-lactide) (PLLA), poly(D,L-lactide), poly(L-lactide-co-caprolactone) (PLLA-co-CL), poly(L-lactide-co-glycolide), or poly(DL-lactide-co-glycolide), (PLGA). The copolymers may be random or block copolymers. The poly(DL-lactide) homopolymer or copolymer component of a polymer formulation can have a constitutional unit weight percentage L-lactide and D-lactide units of 50/50 to 96/4, such as 50/50 or 96/4 poly(DL-lactide). The term “unit” or “constitutional unit” refers to the composition of a monomer as it appears in a polymer. The PLLA-co-CL copolymer can have a weight or mole percentage of caprolactone units of 1 to 10% or more narrowly 1 to 5%, 5 to 10%, 1 to 3%, 3 to 5%, 5 to 8%, or 8 to 10%. PLGA copolymer can have molar percentages of L-lactide or DL-lactide and glycolide units, of 90:10, 75:25, 50:50, 25:75, and 10:90.

The polymer or polymer formulation of a scaffold of the present invention may further include a blend of a PLA polymer with PCL homopolymer; a blend of a PLA homopolymer and a PLA-co-PCL copolymer; and a blend of a PCL homopolymer and a PLA and PCL copolymer. A homopolymer refers to a polymer that is composed of only one type of constitutional unit that is composed of one type of constitutional unit with only trace amounts of other types of units, for example, less than 1 mol % or 0.01 mol %.

In certain aspects of a blend of a PLA homopolymer and a PLA-co-PCL copolymer, the PLA homopolymer is PLLA or PDLLA. The PLA-co-PCL copolymer may be PLLA-co-CL or PDLLA-co CL. The PLA-co-PCL copolymer may be 1 to 20 wt %, 1 to 15 wt %, 5 to 20 wt %, 5 to 15 wt %, 10 to 20 wt %, 15%, 12 to 18 wt %, or 10 to 15 wt % of the blend. The PLA homopolymer may be 80 to 99 wt %, 85 to 99 wt %, 80 to 95 wt %, 85 to 95 wt %, 80 to 90 wt %, 85 wt %, 82 to 88 wt %, or 85 to 90 wt % of the blend. The caprolactone units in the PLA-co-PCL copolymer may be 1 to 10% (wt % or mol %) of the blend, or more narrowly, 1 to 3%, 3 to 5%, 5 to 10%, 2 to 8%, or 3 to 8% of the blend. The random copolymer may be 1% to 50% caprolactone units. Exemplary random copolymers include 95/5 PLA-co-PCL and 70/30 PLA-co-PCL, wherein, for example, 95/5 refers to 95 mol % lactide and 5 mol % caprolactone.

Embodiments of the invention include a scaffold made substantially or completely of the polymer formulation. “Substantially” may correspondent to greater than 90 wt %, greater than 95 wt %, or greater than 99 wt %. The scaffold may have a composition of 90 to 95% or 95 to 99% of the polymer formulation.

The scaffold or the polymer formulation of the scaffold may have a degree of crystallinity of less than 5%, 5 to 20%, 20 to 55%, 20 to 30%, 30 to 40%, 40 to 45%, 45 to 40%, or 50 to 55%. In other embodiments, the scaffold or the polymer formulation of the scaffold may be amorphous or substantially amorphous.

The polymer for a polymer carrier of a therapeutic coating over the scaffold may include PLLA, PDLLA, polyglycolide, PLGA, PCL, or PLA-co-PCL. A drug may be mixed or dispersed throughout the polymer carrier. The drug may be 20 to 80 wt % of the therapeutic layer, or more narrowly, 30 to 70 wt %, 40 to 60 wt %, 45 to 55 wt %, or 50% of the therapeutic layer. Exemplary drugs include rapamycin, everolimus, novolimus, zotarolimus, or biolimus.

The polymer formulation may further include blends with or copolymers of polylactide and polydioxanone, polyethylene oxide, polyethylene glycol, poly(butylene succinate), poly(trimethylene carbonate), poly(butylene succinate), or any combination thereof.

The scaffold and the coating are subjected to stress/strain in localized regions when the stent is crimped and deployed. The inside or concave regions 20, illustrated in FIG. 1, of the bends in the stent pattern or crowns 18 are subjected to high compressive stress and strain when the stent is crimped, but the outside or convex regions 22 of the crowns 18 are subjected to high compressive stress and strain when the stent is deployed. Thus, the coating is susceptible to damage due to deformation of the crowns at the sidewalls, inner surfaces, and outer surfaces of the crowns.

A coating is especially susceptible to damage arising from external forces applied to crimp the scaffold. Generally, stent crimping is the act of affixing the stent to the delivery catheter or delivery balloon so that it remains affixed to the catheter or balloon until the physician desires to deliver the stent at the treatment site. Stent crimping typically involves disposing a stent within an aperture of a crimping device. A force is applied normally to the outer surface of the stent by the walls the aperture to reduce its diameter to a delivery diameter over the catheter or balloon. For example, in a sliding wedge or iris crimper, adjacent pie-piece-shaped sections move inward and twist, much like the leaves in a camera aperture. The sliding wedges impart primarily normal forces, however, as the wedges slide over each other, they impart some tangential force.

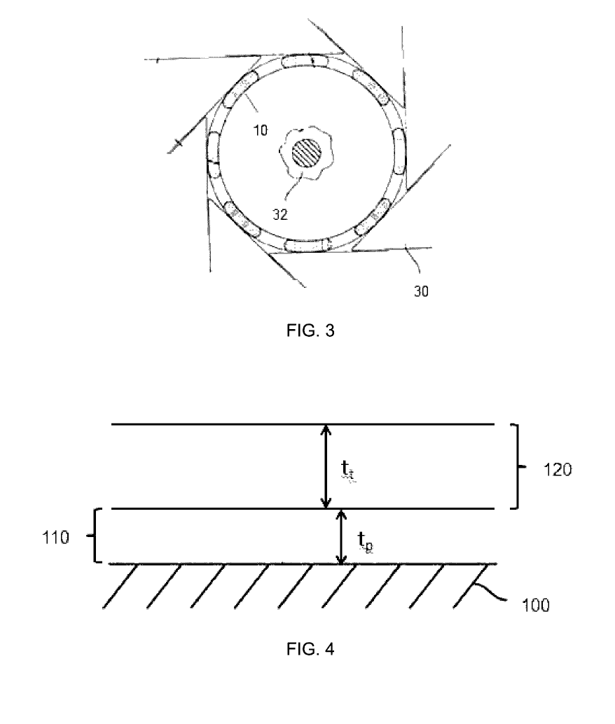

FIG. 3 is a cross-sectional view of a crimper head and scaffold within the aperture of the crimper head showing the orientation of the blades relative to the scaffold when the aperture forms a first diameter and second, smaller diameter, respectively. The scaffold body 10 is disposed between the blades 30. The scaffold 10 is supported on the collapsed balloon 32 of the catheter when it is placed in the crimper head. Then, as the blade edges engage the scaffold the scaffold is lifted off the balloon as shown.

An outer surface of a stent such as a polymer coating is susceptible to damage from the surface of the crimping device applied to the outer surface of the stent. Crimper blades are typically metallic so that metallic blade edges are applying force to a polymer surface of a scaffold or polymer surface of a polymer coating. A softer polymer surface is particularly susceptible damage from a harder metallic surface. Some crimping devices are equipped to dispose a polymer film between the blade edge and the stent surface to reduce potential damage.

Several types of damage to a polymer coating can occur from forces arising from manufacturing and use of a stent. These include flaps, tears, bare spots, and peeling. The degree of damage may be characterized by the amount of surface area of the stent having damage or compromised surface area. The amount of surface area may be expressed as the percent of surface area of the stent or portion of a stent, such as the percent of the outer surface of a stent.

The susceptibility of a coating to damage is expected to be related to the compatibility of the materials of the contacting surfaces, specifically, the compatibility of the coating material to the scaffold material. Compatibility is related to the chemical similarity of the surfaces. The compatibility of a metallic stent surface is to a polymer coating is expected to be significantly lower than the compatibility between two different polymer surfaces. The compatibility between two chemically similar polymer surfaces is expected to be higher than between chemically different polymer surfaces.

As described in the examples herein, the inventors observed significant coating damage due to crimping on the outer surface of crimped bioresorbable polymer stents. The stents included a scaffold composed of a blend of PLLA homopolymer and PLLA-co-PCL copolymer. The PLLA homopolymers was about 85 wt % of the blend and the overall wt % of the L-lactide units in the blend, including the homopolymer and copolymer, was between 93 and 98 wt %. The stent also included a coating directly on the scaffold surface which was 50 wt % 50/50 PDLLA and 50 wt % everolimus. The polymer of the coating and scaffold are chemical similar and expected to be highly compatible. Coating defects that were observed included lifted coating, peeling and voids. Filmless crimpers were employed for crimping all samples.

Aspects of the present invention are directed at reducing or eliminating damage to polylactide based drug and polymer coatings on scaffolds. The aspects have been shown in particular to reduce damage to coatings from external forces during crimping of the stents. The invention includes an intermediate layer or primer layer between the scaffold and a therapeutic layer. As the therapeutic layer incorporates immiscible drug as well as polymer, such as PDLLA, the primer layer may provide the intermediate properties between a scaffold polymer (such as PLLA) and a polymer and drug layer (such as drug and PDLLA) with a high weight percent of drug. The aspects may also reduce or eliminate damage resulting from deformation of structural elements during crimping and deployment.

A stent fabricated according to the various aspects of the invention may have reduced damage to the coating or no damage to the coating. The amount of damage may be correspond to the percentage of damaged surface area or which may also be referred to as compromised surface area. In one aspect, the compromised surface are of a surface, such as the outer surface, of the stent may be less than 10%, 1 to 10%, less than 5%, 1 to 5%, 2 to 5%, or 4 to 6%. In other aspects, the stent may have a surface such as the outer surface of the crimped stent that is free of compromised surface area.

Certain aspects of the invention include a method of fabricating a stent that include providing a scaffold comprising a polymer formulation including a first polylactide polymer or scaffold polymer. The method further includes forming a primer layer on a surface of the scaffold, wherein the primer layer includes a second polylactide polymer or primer polymer and the primer layer is free of a therapeutic agent. The scaffold with the primer layer may then be thermally treated or processed to condition the scaffold. The thermal treatment improves the mechanical stability and adhesion of the primer layer to the scaffold. In particular, it is believed that the thermal treatment increases the resistance to fracture of the primer layer and the resistance to coating damage related to loss of adhesion.

After the thermal treatment, the method further includes forming a therapeutic layer over the primer layer. The therapeutic layer includes the second polylactide polymer and a drug. The scaffold having the therapeutic and primer layers may be crimped from a fabricated diameter to a reduced diameter for delivery into a vascular lumen. The primer layer improves adhesion of the therapeutic layer to the scaffold and reduces or prevents crimping-induced damage to a surface of the therapeutic layer.

FIG. 4 depicts a cross-section of a strut 100 of a scaffold with a primer layer 110 over strut 100 and a therapeutic layer 120 over primer layer 110. Primer layer 110 has a thickness tₚ and the therapeutic layer 120 has thickness tₜ.

In these aspects, the polylactide polymer of the primer layer, the second polylactide polymer, is the same as the polylactide polymer of the therapeutic layer. Thus, the composition of the primer layer differs from therapeutic layer at least in that the primer layer is free of the drug in the therapeutic layer. Thus, the primer layer may be more compatible with the first polylactide polymer of the scaffold which is different from the second polylactide polymer of the primer and therapeutic layers.

In an exemplary embodiment, the polymer formulation of the scaffold is PLLA. In another embodiment, the polymer formulation is PLLA-co-PCL. In another embodiment, the polymer formulation of the scaffold is any one of the aspects described herein of a blend of PLLA and PLLA-co-PCL. The primer polymer in any of these embodiments may be 50/50 PDLLA. The therapeutic layer may be 50/50 PDLLA and a drug in any of the ranges disclosed herein.

In other embodiments, the scaffold may be made of a metal. In such embodiments, the primer layer improves adhesion of the therapeutic layer to the metal scaffold and reduces or prevents crimping-induced damage to a surface of the therapeutic layer.

The primer and therapeutic layers may be formed using various solution processing methods described above. To form the primer layer, a coating composition including the second polylactide polymer dissolved in a solvent is applied to the surface of the scaffold, followed by removal of the solvent. The coating composition is free of drug. Exemplary solvents include acetone, chloroform, methyl ethyl ketone (MEK), and cyclohexanone combined with acetone. The primer layer may be formed in several passes of application of the coating composition as described above. In exemplary embodiments, a primer layer of any thickness may be formed in 1 to 6 passes, or more narrowly, 1 to 3 passes or 3 to 6 passes.

In the case of a therapeutic layer, a coating composition includes the second polylactide polymer dissolved in a solvent and a drug suspended or dissolved in a solvent. The solvent may be the same or different from the coating composition of the primer layer. The solvent may include any of those listed above.

It is desirable for the thickness of the primer layer to be small to minimize the radial profile of the stent as a whole. The thickness of the primer layer may be 0.2 to 3 microns, or more narrowly, 0.1 to 0.5 micron, 0.1 to 1 micron, 0.5 to 1 micron, 1 to 2 microns, 1 to 3 microns, or 2 to 3 microns.

The thickness of the therapeutic layer may be 1 to 5 microns, or more narrowly, 1 to 2 microns, 2 to 3 microns, 3 to 5 microns, 4 to 5 microns or greater than 5 microns. The thickness of the primer layer and the therapeutic layer combined may be 3 to 8 microns, or more narrowly, 3 to 4 microns, 4 to 6 microns, or 6 to 8 microns.

The primer layer and therapeutic layer may be applied on all surfaces of the scaffold, the abluminal or outer surface, luminal or inner surface, and sidewall surfaces. In some aspects, the primer layer, therapeutic layer, or both may be applied exclusively on selected surfaces. For example, a layer may be applied exclusively to the abluminal surface, luminal surface, or both.

Additionally, the thickness of a layer or layers on a particular surface may not be uniform across the surface. Also, the thickness or average thickness for the abluminal surface, luminal surface, and sidewall surface may be different. For example, the abluminal thickness may be greater than the luminal thickness which may be greater than the sidewall thickness. A thickness of a coating may refer to a thickness at a particular region or point on a surface or an average thickness on a particular surface or an average thickness over all the surfaces.

After each pass of applying a coating composition during the coating process, the coating may be subjected to a drying step that includes heating the stent to a temperature above ambient to remove solvent. The temperature may, for example, be 40 to 50 deg C. or greater than 40 deg C. and less than a Tg of first polylactide polymer, second polylactide polymer or both.

After the final pass in forming the primer layer or the therapeutic layer, the coating may include residual solvent. The coating may or may not be subjected to a further solvent removal step that includes heating the scaffold, such as baking in an oven, at a mild temperature for a suitable duration of time (e.g., 30 min to 4 hr) or by the application of warm air. The mild temperature may be less than the Tg of the first polylactide polymer, second polymer lactide polymer, or both. For a polylactide scaffold polymer the solvent removal temperature may be 40 to 50 deg C.

At the completion of forming the primer layer, the prime may have less than 5 wt % residual solvent, or more narrowly, 0.1 to 1 wt %, 1 to 5 wt %, 1 to 2 wt %, or 2 to 3 wt %. At the completion of forming the therapeutic layer, the therapeutic layer may have less than 5 wt % residual solvent, or more narrowly, 0.1 to 1 wt %, 1 to 5 wt %, 1 to 2 wt %, or 2 to 3 wt %.

In certain aspects, the thermal treatment comprises increasing a temperature of the scaffold with the primer layer above a Tg and below a melting temperature (Tm) of the polymer formulation followed by reducing the temperature below the Tg. In one aspect, the temperature of the scaffold is increased to 5 to 10 deg or 10 to 20 deg C. above the Tg and maintained for 10 or 20 minutes or 5 and 10 minutes. After reducing the temperature below the Tg, the therapeutic layer may be applied over the primer layer. In exemplary aspects, the temperature may be 70 to 90 deg C., 70 to 75 deg C., 75 to 80 deg C., 80 deg C., 80 to 85 deg C., or 85 to 90 deg C. These temperature ranges may, for example, apply to a scaffold polymer having 80 to 100 wt % PLA, PLLA, or PDLLA homopolymer or 80 to 100 wt % lactide content. The thermal treatment is disclosed in detail in U.S. Patent Application No. 62/052,393.

Thermal treatment conditions the scaffold and primer coating in addition to removing residual solvent. The conditioning of the scaffold includes reversal of physical aging of the scaffold that has occurred during the manufacturing process. The conditioning includes modification of scaffold properties. Modification of the scaffold may include decrease in density of the scaffold polymer, increase in elongation at break of the scaffold polymer, decrease in modulus of the scaffold polymer, increase in radial strength of the scaffold, increase in expansion capability of the scaffold, reduced damage to the scaffold at crimping, and any combination thereof.

In further aspects of the invention, a method of fabricating a may include providing a scaffold including a polymer formulation including a first polylactide polymer. The method may further include forming a primer layer on a surface of the scaffold. The primer layer includes a second polylactide polymer and an amount of drug. The amount of drug may be less than 10 wt % of the primer layer or 0.1 to 10 wt % of the primer layer.

The method further includes thermally treating the scaffold with the primer layer to condition the scaffold. After the thermal treatment, a therapeutic layer may be formed over the primer layer. The therapeutic layer includes the second polylactide polymer and an amount of the drug greater than the amount of drug in the primer layer. In one aspect, the amount of drug in the therapeutic layer is greater than 20 wt %, 20 to 80 wt %, or 40 to 60 wt % of the therapeutic layer.

The method further includes crimping the scaffold having the primer and therapeutic layers from a fabricated diameter to a reduced diameter for delivery into a vascular lumen.

The scaffold having the therapeutic and primer layers may be crimped from a fabricated diameter to a reduced diameter for delivery into a vascular lumen. Rather than being free of drug, the primer layer in these aspects includes a smaller amount of drug than the therapeutic layer. Since it has a smaller amount of drug, the primer layer is more compatible with the scaffold than the therapeutic layer. Thus, the primer layer may improve adhesion of the therapeutic layer to the scaffold and reduces or prevents crimping-induced damage to a surface of the coating.

In other embodiments, the scaffold may be made of a metal. In such embodiments, the primer layer with a lesser amount of drug improves adhesion of the therapeutic layer with a greater amount of drug to the metal scaffold and reduces or prevents crimping-induced damage to an outer surface of the coating on the metal scaffold.

Another aspect of the invention include includes a method of fabricating a stent including providing a scaffold including a polymer formulation including a scaffold polylactide polymer. The method further includes forming a primer layer on a surface of the scaffold. The primer layer may include a primer polylactide polymer and a drug and the drug is less than 10 wt % of the primer layer. The scaffold with the primer layer may be thermally treated to condition the scaffold, coating, or both. After the thermal treatment a therapeutic layer may be formed over the primer layer. The therapeutic layer may include a therapeutic polylactide polymer and an amount of the drug greater than the amount of drug in the primer layer. In one aspect, the amount of drug in the therapeutic layer is greater than 20 wt % or 20 to 80 wt % of the therapeutic layer.

The method further includes crimping the scaffold having the primer and therapeutic layers from a fabricated diameter to a reduced diameter for delivery into a vascular lumen. The primer layer improves adhesion of the therapeutic layer to the scaffold and reduces or prevents crimping-induced damage to a surface of the therapeutic layer.

In one aspect, the primer polylactide polymer and the therapeutic polylactide polymer are different. In an exemplary aspect the scaffold polylactide polymer is any one of the aspects of PLLA, PLLA-co-PCL, or blend of PLLA and PLLA-co-PCL. The exemplary aspect further includes a primer polylactide polymer of 96/4 PDLLA or PLLA and a therapeutic polylactide polymer of 50/50 PDLLA.

In other aspects, the primer and therapeutic layer polymers are the same, such as 50/50 PDLLA

In one aspect, the amount of drug in the primer layer is 0.1 to 10 wt %. In another aspect the primer layer is free of drug.

In one aspect, the amount of drug in the therapeutic layer is 40 to 60 wt %.

In further aspects, adhesion may be further improved via mechanisms including, but not limited to, roughening of the scaffold surface, better chemical compatibility between the therapeutic coating, and the primer, and lower solvent concentrations at the interfaces.

### Examples

A first set of stents was fabricated having a scaffold composed of a blend of PLLA homopolymer and PLLA-co-PCL copolymer. The scaffold was 85 wt % PLLA and 15 wt % copolymer. The PLLA-co-PCL copolymer was 70 mol % PLLA based on L-lactide. The stents had a coating composed of 50/50 PDLLA and everolimus. The coating composition was 50 wt % PDLLA and 50 wt % everolimus.

A second set of stents were fabricated according to embodiments of the present invention. The stents had the same scaffold as the first set. Unlike the first set, a thin primer layer of PDLLA was formed by spraying a PDLLA/acetone composition to the scaffold surface. After removal of solvent, a thermal process was performed to thermally condition the scaffold, the primer layer, and remove residual solvent.

Stents without the primer layer and no thermal treatment are referred to as untreated stents (first set) and stents with the primer layer with thermal treatment are referred to as treated stents (second set).

Both sets of stents were crimped with a filmless iris crimper from a fabricated diameter to crimped diameter over a 3.0×18 mm catheter balloon. The stents were then expanded or deployed by the balloon to the nominal diameter (3.0 mm in this case) in 37 deg C. water.

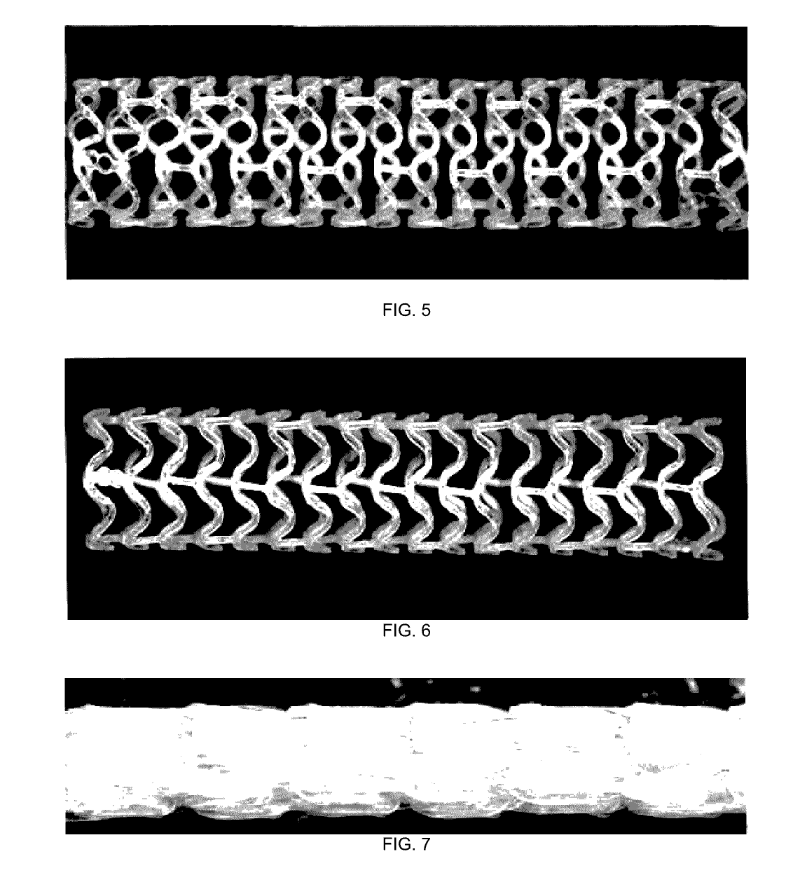

FIGS. 5 and 6 demonstrate the improved adhesion of treated stents versus untreated stents after crimping and deploying. FIG. 5 depicts a deployed untreated stent at time=0, i.e., right after deployment. Multiple areas are shown where the coating failed to adhere along the length of the stent. FIG. 6 depicts a deployed treated stent. The reduced coating failure or damage of the surface demonstrates a significant improvement over the untreated stent. The stent shown was soaked in a 25 deg C. water bath for 24 hours prior to testing for radial strength.

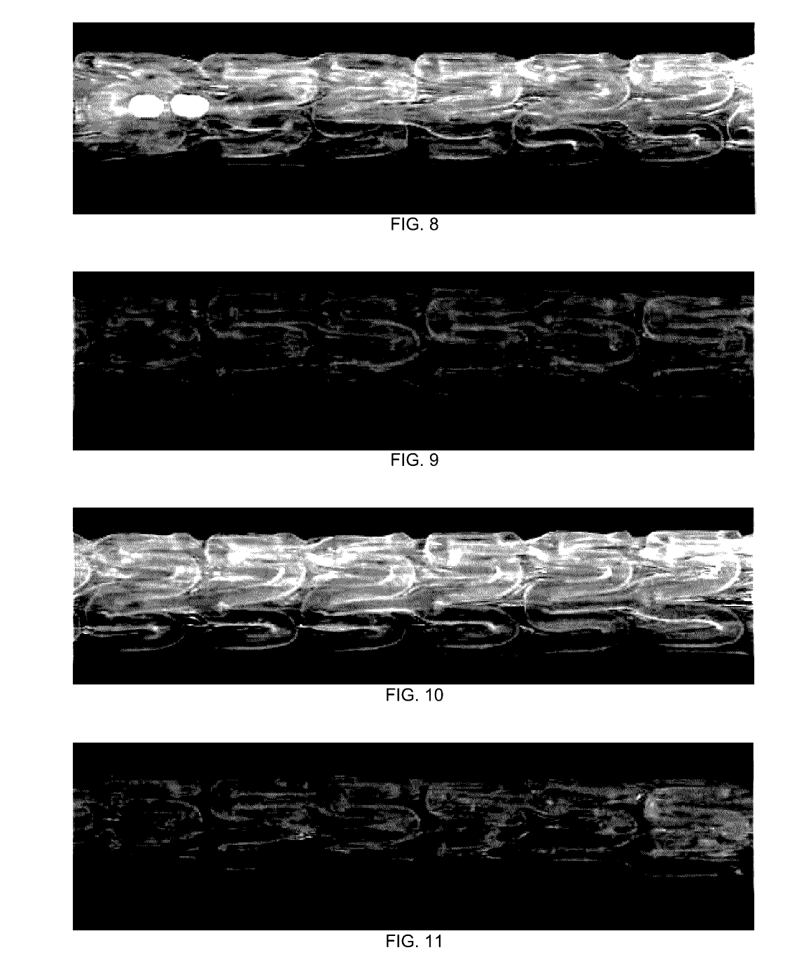

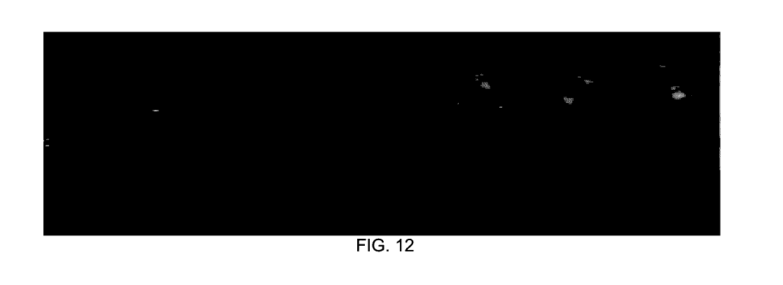

FIGS. 7-12 depict close-ups of the distal, middle, and proximal sections of untreated and treated crimped scaffolds, showing that the coating damage is a function of crimping rather than deployment. FIG. 7 depicts a distal end of a crimped untreated stent having multiple areas where coating was compromised upon crimping. FIG. 8 depicts a distal end of a crimped treated stent showing less coating damage than the untreated stent. FIG. 9 depicts a middle section of an untreated stent with multiple areas where the coating was compromised upon crimping. FIG. 10 depicts a middle section of a crimped treated stent showing less coating damage than the untreated stent. FIG. 11 depicts an untreated stent showing a proximal ring section as crimped before a final sheath is placed over the stent having multiple areas where coating has been compromised. FIG. 12 depicts a proximal end of a crimped treated stent showing less coating damage than the untreated stent.

The drug in the aspects of the present invention includes an antiproliferative, anti-inflammatory or immune modulating, anti-migratory, anti-thrombotic or other pro-healing agent or a combination thereof. The anti-proliferative agent can be a natural proteineous agent such as a cytotoxin or a synthetic molecule or other substances such as actinomycin D, or derivatives and analogs thereof (manufactured by Sigma-Aldrich 1001 West Saint Paul Avenue, Milwaukee, Wis. 53233; or COSMEGEN available from Merck) (synonyms of actinomycin D include dactinomycin, actinomycin IV, actinomycin I1, actinomycin X1, and actinomycin C1), all taxoids such as taxols, docetaxel, and paclitaxel, paclitaxel derivatives, all olimus drugs such as macrolide antibiotics, rapamycin, everolimus, novolimus, myolimus, deforolimus, umirolimus, biolimus, merilimus, temsirolimus structural derivatives and functional analogues of rapamycin, structural derivatives and functional analogues of everolimus, FKBP-12 mediated mTOR inhibitors, biolimus, perfenidone, prodrugs thereof, co-drugs thereof, and combinations thereof. Representative rapamycin derivatives include 40-O-(3-hydroxy)propyl-rapamycin, 40-O-[2-(2-hydroxy)ethoxy]ethyl-rapamycin, or 40-O-tetrazole-rapamycin, 40-epi-(N1-tetrazolyl)-rapamycin (ABT-578 manufactured by Abbott Laboratories, Abbott Park, Ill.), prodrugs thereof, co-drugs thereof, and combinations thereof.

The anti-inflammatory agent can be a steroidal anti-inflammatory agent, a nonsteroidal anti-inflammatory agent, or a combination thereof. In some embodiments, anti-inflammatory drugs include, but are not limited to, novolimus, myolimus, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus, prodrugs thereof, co-drugs thereof, and combinations thereof.

These agents can also have anti-proliferative and/or anti-inflammatory properties or can have other properties such as antineoplastic, antiplatelet, anti-coagulant, anti-fibrin, antithrombonic, antimitotic, antibiotic, antiallergic, antioxidant as well as cystostatic agents. Other active agents which are currently available or that may be developed in the future are equally applicable.

The “glass transition temperature,” Tg, is the temperature at which the amorphous domains of a polymer change from a brittle vitreous state to a solid deformable or ductile state at atmospheric pressure. In other words, the Tg corresponds to the temperature where the onset of segmental motion in the chains of the polymer occurs. When an amorphous or semi-crystalline polymer is exposed to an increasing temperature, the coefficient of expansion and the heat capacity of the polymer both increase as the temperature is raised, indicating increased molecular motion. As the temperature is increased, the heat capacity increases. The increasing heat capacity corresponds to an increase in heat dissipation through movement. Tg of a given polymer can be dependent on the heating rate and can be influenced by the thermal history of the polymer as well as its degree of crystallinity. Furthermore, the chemical structure of the polymer heavily influences the glass transition by affecting mobility. The Tg can be determined as the approximate midpoint of a temperature range over which the glass transition takes place. [ASTM D883-90]. The most frequently used definition of Tg uses the energy release on heating in differential scanning calorimetry (DSC). As used herein, the Tg refers to a glass transition temperature as measured by differential scanning calorimetry (DSC) at a 20° C./min heating rate. Unless stated otherwise, values for “Tg” refer to an upper limit for Tg (E.g., for poly(L-lactide) and the Tg when the material is dry. Poly(L-lactide) has a glass transition temperature range of between about 55 to 60 Deg. C. “Tg” for poly(L-lactide), for purposes of this disclosure, Tg is 60 Deg. C), or up to 65 Deg. C. for a strain hardened tube. The glass transition temperature is a function of chain flexibility. The glass transition occurs when there is enough vibrational (thermal) energy in the system to create sufficient free-volume to permit sequences of 6-10 main-chain carbons to move together as a unit. At this point, the mechanical behavior of the polymer changes from rigid and brittle to tough and leathery.

The “melting temperature” (Tm) is the temperature at which a material changes from solid to liquid state. In polymers, Tm is the peak temperature at which a semicrystalline phase melts into an amorphous state. Such a melting process usually takes place within a relative narrow range (<20° C.), thus it is acceptable to report Tm as a single value.

“Modulus” may be defined as the ratio of a component of stress or force per unit area applied to a material divided by the strain along an axis of applied force that result from the applied force. For example, a material has both a tensile and a compressive modulus.

“Toughness”, or “fracture toughness” is the amount of energy absorbed prior to fracture, or equivalently, the amount of work required to fracture a material. One measure of toughness is the area under a stress-strain curve from zero strain to the strain at fracture. The stress is proportional to the tensile force on the material and the strain is proportional to its length. The area under the curve then is proportional to the integral of the force over the distance the polymer stretches before breaking. This integral is the work (energy) required to break the sample. The toughness is a measure of the energy a sample can absorb before it breaks. There is a difference between toughness and strength. A material that is strong, but not tough is said to be brittle. Brittle materials are strong, but cannot deform very much before breaking.

The “degree of crystallinity” may be expressed in terms of, w_{c} (mass fraction), φ_{c} (volume fraction) and refers to mass fraction or volume fraction of crystalline phase in a sample of polymer. The mass-fraction and the volume-fraction degrees of crystallinity are related by the equation, w_{c}=φ_{c} ρ/ρ_{c}, where ρ and ρ_{c} are the mass concentrations (mass densities) of the entire sample and of the crystalline phase, respectively. The degree of crystallinity can be determined by several experimental techniques. Among the most commonly used are: (i) x-ray diffraction, (ii) calorimetry (DSC), (iii) mass density measurements, (iv) infrared spectroscopy (IR), (v) solid-state NMR spectroscopy, and (vi) vapor permeability. Unless stated otherwise, throughout this description a degree of crystallinity given for a polymer is expressed as a percentage (%) of crystallinity and expressed as a mass or volume fraction. Unless stated otherwise throughout this description a degree of crystallinity given for a polymer composition is expressed as a percentage (%) of crystallinity and expressed as a mass fraction. Measurements of crystallinity may also be determined from a modified method of differential scanning calorimetry (DSC), e.g., over a temperature range of 30 Deg. C. to 150 Deg. C, with modulation amplitude of 0.5° C. and heat rate of 6° C./minute and duration of 1 minute.

“Amorphous” or “substantially amorphous” means no greater than, or less than 5% crystallinity, or not more than 1%, 2% or 4% crystallinity.

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

What is claimed is:

1. A method of fabricating a stent comprising:
providing a scaffold comprising a polymer formulation including a first polylactide polymer;
forming a primer layer on a surface of the scaffold, wherein the primer layer comprises a second polylactide polymer and the primer layer is free of a therapeutic agent and;
thermally treating the scaffold with the primer layer to condition the scaffold;
forming a therapeutic layer over the primer layer, wherein the therapeutic layer comprises the second polylactide polymer and a drug; and
crimping the scaffold having the therapeutic and primer layers from a fabricated diameter to a reduced diameter for delivery into a vascular lumen, wherein the primer layer improves adhesion of the therapeutic layer to the scaffold and reduces or prevents damage to the therapeutic layer.

2. The method of claim 1, wherein the therapeutic layer comprises damage over less than 2% of a surface area of an outer surface of the crimped stent and wherein the damage comprises flaps, tears, bare spots, and/or peeling.

3. The method of claim 1, wherein the crimping comprises applying a radial inward force on an outer surface of the stent with metallic surfaces to reduce a diameter of the stent to the reduced diameter.

4. The method of claim 1, wherein to condition the scaffold comprises modification of scaffold properties that include a decrease in density of the scaffold polymer, an increase in elongation at break of the scaffold polymer, a decrease in modulus of the scaffold polymer, an increase in radial strength of the scaffold, an increase in expansion capability of the scaffold, a reduction in damage to the scaffold at crimping, or any combination thereof.

5. The method of claim 1, wherein the polymer formulation is selected from the group consisting of poly(L-lactide) (PLLA), a copolymer of PLLA and polycaprolactone, a blend of PLLA and a copolymer of PLLA and PCL.

6. The method of claim 5, wherein the polymer formulation comprises a total CL content of 2 to 8 wt %.

7. The method of claim 1, wherein the thermal treatment comprises increasing a temperature of the stent above a glass transition temperature (Tg) and below a melting temperature (Tm) of the polymer formulation followed by reducing the temperature below the Tg.

8. The method of claim 1, wherein the thermal treatment comprises increasing the temperature of the scaffold to 10 to 20 deg C. above the Tg of the polymer formulation and maintaining the increased temperature for 10 to 20 minutes or 5 to 10 minutes followed by reducing the temperature to below the Tg.

9. The method of claim 1, wherein the second polylactide polymer is 50/50 poly(DL-lactide).

10. The method of claim 1, wherein the drug is selected from the group consisting of everolimus, rapamycin, novolimus, zotarolimus, and biolimus.

11. The method of claim 1, wherein a thickness of the primer layer is 0.2 to 2 microns and a thickness of the therapeutic layer is 1 to 3 microns.

12. A method of fabricating a stent comprising:
providing a scaffold comprising a polymer formulation including a first polylactide polymer;
forming a primer layer on a surface of the scaffold, wherein the primer layer comprises a second polylactide polymer and a drug and the drug is 0.1 to 10 wt % of the primer layer;
thermally treating the scaffold with the primer layer to condition the scaffold;
forming a therapeutic layer over the primer layer, wherein the therapeutic layer comprises the second polylactide polymer and a drug and the drug is greater than 20 wt % of the therapeutic layer; and
crimping the scaffold having the primer and therapeutic layers from a fabricated diameter to a reduced diameter for delivery into a vascular lumen, wherein the primer layer improves adhesion of the therapeutic layer to the scaffold and reduces or prevents damage to the therapeutic layer.

13. The method of claim 12, wherein the drug is 40 to 60 wt % of the therapeutic layer.

14. The method of claim 12, wherein the therapeutic layer comprises damage over less than 2% of a surface area of an outer surface of the crimped stent and wherein the damage comprises flaps, tears, bare spots, and/or peeling.

15. The method of claim 12, wherein the crimping comprises applying a radial inward force on an outer surface of the stent with metallic surfaces to reduce a diameter of the stent to the reduced diameter.

16. The method of claim 12, wherein to condition the scaffold comprises modification of scaffold properties that include a decrease in density of the scaffold polymer, an increase in elongation at break of the scaffold polymer, a decrease in modulus of the scaffold polymer, an increase in radial strength of the scaffold, an increase in expansion capability of the scaffold, a reduction in damage to the scaffold at crimping, or any combination thereof.

17. The method of claim 12, wherein the polymer formulation is selected from the group consisting of poly(L-lactide) (PLLA), a copolymer of PLLA and polycaprolactone, a blend of PLLA and a copolymer of PLLA and PCL.

18. The method of claim 17, wherein the polymer formulation comprises a total CL content of 2 to 8 wt %.

19. The method of claim 12, wherein the thermal treatment comprises increasing a temperature of the stent above a glass transition temperature (Tg) and below a melting temperature (Tm) of the polymer formulation followed by reducing the temperature below the Tg.

20. The method of claim 12, wherein the thermal treatment comprises increasing the temperature of the scaffold to 10 to 20 deg C. above the Tg of the polymer formulation and maintaining the increased temperature for 10 to 20 minutes or 5 to 10 minutes followed by reducing the temperature to below the Tg.

21. The method of claim 12, wherein the second polylactide polymer is 50/50 poly(DL-lactide).

22. The method of claim 12, wherein the drug is selected from the group consisting of everolimus, rapamycin, novolimus, zotarolimus, and biolimus.

23. The method of claim 12, wherein a thickness of the primer layer is 0.2 to 2 microns and a thickness of the therapeutic layer is 1 to 3 microns.
